(19) United States
(12) Patent Application Publication
(10) Pub. No.: US 2022/0105219 A1
(43) Pub. Date: Apr. 7, 2022

(54) Door Handle Sanitizer

(71) Applicants: John Thompson, New York, NY (US); Laurie Richards, Miami Beach, FL (US); Daniel Richards, Miami Beach, FL (US)

(72) Inventors: John Thompson, New York, NY (US); Laurie Richards, Miami Beach, FL (US); Daniel Richards, Miami Beach, FL (US)

(21) Appl. No.: 17/494,731

(22) Filed: Oct. 5, 2021

Related U.S. Application Data
(60) Provisional application No. 63/087,506, filed on Oct. 5, 2020.

(51) Int. Cl. A61L 2/24; A61L 2/22

(52) U.S. Cl. CPC A61L 2/24; A61L 2202/15; A61L 2202/14; A61L 2/22

(57) ABSTRACT

A door handle sanitizer includes a housing that is mountable to a door and an access lid pivotally attached to the housing. An interior volume of the housing includes a dispensing mechanism having a connector disposed within the interior volume that is configured to removably receive a sanitizer refill container, and a nozzle configured to dispense a sanitizing material onto the door handle. A power supply is operably connected to a motion sensor, a timer, indicator lights, a dispensing mechanism actuator, and a control circuit. The control circuit includes control switches that allow for customization of the door handle sanitizer's spray frequency. If a timer switch is set to a timer interval, then the control circuit activates the dispenser in an interval corresponding to the selected interval. If a motion sensor switch is set to an on position, then the control circuit activates the dispensing mechanism actuator when the motion sensor detects motion.

### CROSS REFERENCE TO RELATED APPLICATIONS

This application claims the benefit of U.S. Provisional Application No. 63/087,506, filed on Oct. 5, 2020. The above identified patent application is herein incorporated by reference in its entirety to provide continuity of disclosure.

### BACKGROUND OF THE INVENTION

The present invention relates to an automated door handle sanitizing device. More specifically, the present invention provides a door handle sanitizer with selectable operational modes, sensors, and usage monitoring.

Public spaces and common contact areas can harbor germs, bacteria, viral particles, and other unwanted contaminants. Individuals often seek to limit their exposure to germs and other harmful elements on public surfaces, such as door handles for example, in order to minimize the risk of contracting an illness. This can include washing and sanitizing one's hands frequently. However, individuals do not always have a sanitizing source at hand. Further, even when personal precautions are taken, factors outside of an individual's control can cause them to have unwanted contact with such contaminants. Door handles in public spaces or private spaces with many occupants are frequently contacted by many individuals, but usually are not sanitized more than once a day at most during a typical cleaning. Many places do not have the resources to manually sanitize the door handle frequently enough to prevent the buildup of contaminates. When not properly cleaned, it is common for bacteria and other germs to be transmitted from the door handle to the individual's hand. If the individual then contacts their face or other sensitive area before they have a chance to sanitize their hands, it is likely that germs will be transmitted into their body, potentially causing illness to occur. Without proper, visible sanitization protocols, individuals may be uncomfortable entering certain spaces. In order to address these concerns, it is desirable to provide an automated door handle sanitizer, particularly one that can be programmed to operate in different modes depending on the preference of the user.

Devices have been disclosed in the known art that relate to automated door handle devices. These include devices that have been patented and disclosed in patent application publications. However, the devices in the known art have several drawbacks. For example, these devices typically only have one mode of operation and lack the ability to be customized for one or both of timed dispensing and motion sensed dispensing, whilst also providing an option to pause operations whilst the surrounding room is dark and whilst a hand/object is near. Further, these systems do not include usage monitors that provide notifications when power supplies or canister levels are getting low, and confirmation that a handle has been cleaned through a confirmation indicator.

In light of the devices disclosed in the known art, it is submitted that the present invention substantially diverges in design elements from the known art and consequently it is clear that there is a need in the art for an improvement to existing door handle sanitizing devices. In this regard the present invention substantially fulfills these needs.

### SUMMARY OF THE INVENTION

The present invention provides a door handle sanitizer wherein the same can be utilized for automatically sanitizing a door handle in time intervals and/or in response to motion detected proximal to the door handle. The door handle sanitizer includes a housing having a rear surface that is mountable to a door, via adhesive or other suitable fasteners. An access lid is pivotally attached to the housing and is configured to move between a closed position covering an interior volume of the housing and open position exposing the interior volume of the housing. A dispensing mechanism includes a connector disposed within the interior volume which is configured to removably secure an open end of a sanitizer refill container thereto, and also includes a nozzle configured to dispense a material from the sanitizer refill container when the dispensing mechanism is activated.

A battery-operated power supply is operably connected to a motion sensor, a timer, a plurality of indicator lights, an actuator of the dispensing mechanism, and a control circuit. The control circuit includes a plurality of control switches, a processor, a non-transitory computer readable medium operatively connected to the processor, and a logic stored in the non-transitory computer readable medium that, when executed by the processor, causes the system to perform a method of operation. If a timer switch is set to a timer interval, then the control circuit activates the dispensing mechanism actuator in an interval corresponding to the selected time interval. Further, if a motion sensor switch is set to an on position, then the control circuit activates the dispensing mechanism actuator when the motion sensor detects motion.

One object of the present invention is to provide a door handle sanitizer that can be customized to dispense sanitizing material onto the door handle when motion is detected, after a certain time interval elapses, or when both occur.

Another object of the present invention is to provide a door handle sanitizer with a replaceable refill container of sanitizing material, such that the container can be easily replaced with a new or refilled container when it is empty.

A further object of the present invention is to provide a door handle sanitizer with various indicator lights for conveying information to the user, including battery status, sanitizing material level status, and dispenser activation status, for example.

Another object of the present invention is to provide an option to allow the device to pause operations whilst the surrounding room is dark.

Another object of the present invention is to provide an option to pause dispensing whilst a hand or object is close to the sensor located by the dispensing mechanism

Still a further object of the present invention is to provide a door handle sanitizer that can monitor overall usage of the expensing mechanism over time.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the exterior of an embodiment of the door handle sanitizer.

FIG. 2 shows a perspective view of the interior of an embodiment of the door handle sanitizer.

FIG. 3 shows a front elevation view of an embodiment of the door handle sanitizer attached to a door and sanitizing the door handle.

FIG. 4 shows a block diagram of the various components of an embodiment of the door handle sanitizer.

### DETAILED DESCRIPTION OF THE INVENTION

According to some embodiments, the operations, techniques, and/or components described herein can be implemented as (i) a special-purpose computing device having specialized hardware and a logic hardwired into the computing device to persistently perform the disclosed operations and/or techniques or (ii) a logic that is implementable on an electronic device having a general purpose hardware processor to execute the logic and a computer-readable medium, e.g. a memory, wherein implementation of the logic by the processor on the electronic device provides the electronic device with the function of a special-purpose computing device.

In the interests of economy, the present disclosure refers to “a computer-readable medium,” “a processor,” and so on. However, this should not be read as limiting in any way as the present disclosure contemplates embodiments of the present invention utilizing “one or more computer-readable media,” “one or more processors,” and so on. Unless specifically limited to a single unit, “a” is intended to be equivalent to “one or more” throughout the present disclosure. As used herein, the term “processor” can refer to any electronic circuit which performs operations on some external source. As used herein, the term “logic” can include any combination of computer software instructions, integrated circuit-based logic, gates, switch or junction-based logic gates, etc. As used herein, the term “memory” can refer to any non-transitory computer readable medium, including but not limited to simple circuit states via logic gates or switch positions, as well as solid state computer readable storage.

According to some embodiments, the operations, techniques, and/or components described herein can be implemented by an electronic device, which can include any combination of digital and analogue circuitry, as well as one or more special-purpose computing devices. The special-purpose computing devices can be hard-wired to perform the operations, techniques, and/or components described herein, or can include digital electronic devices such as one or more application-specific integrated circuits (ASICs) or field programmable gate arrays (FPGAs) that are persistently programmed to perform the operations, techniques and/or components described herein, or can include one or more general purpose hardware processors programmed to perform such features of the present disclosure pursuant to program instructions in firmware, memory, other storage, or a combination. Such special-purpose computing devices can also combine custom hard-wired logic, ASICs, or FPGAs with custom programming to accomplish the technique and other features of the present disclosure. The special-purpose computing devices can be desktop computer systems, portable computer systems, handheld devices, networking devices, or any other device that incorporates hard-wired and/or program logic to implement the techniques and other features of the present disclosure.

Reference is made herein to the attached drawings. Like reference numerals are used throughout the drawings to depict like or similar elements of the wireless water control system. For the purposes of presenting a brief and clear description of the present invention, a preferred embodiment will be discussed as used for providing a door handle sanitizer that can be programmed to dispense sanitizing material onto a door handle in time intervals, in response to motion sense proximal to the door handle, or both. Further, the door handle sanitizer is controllable via internal switch mechanisms or via a wireless connection with a remote device. The figures are intended for representative purposes only and should not be considered to be limiting in any respect.

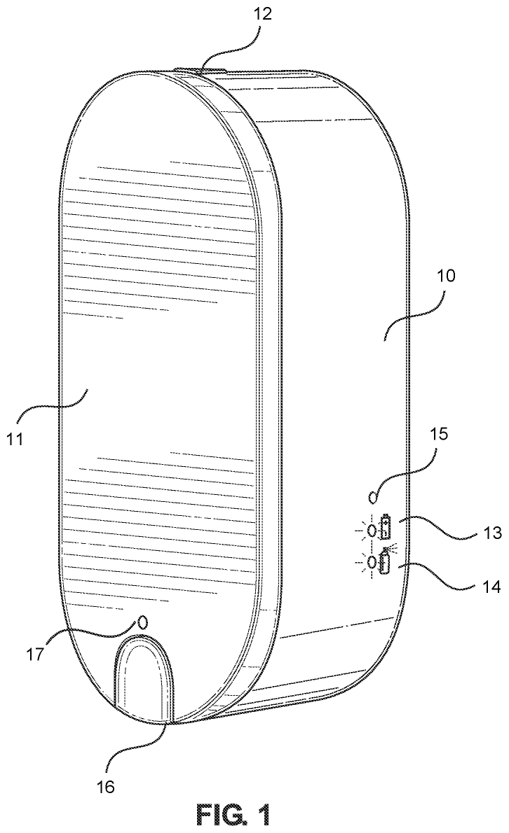

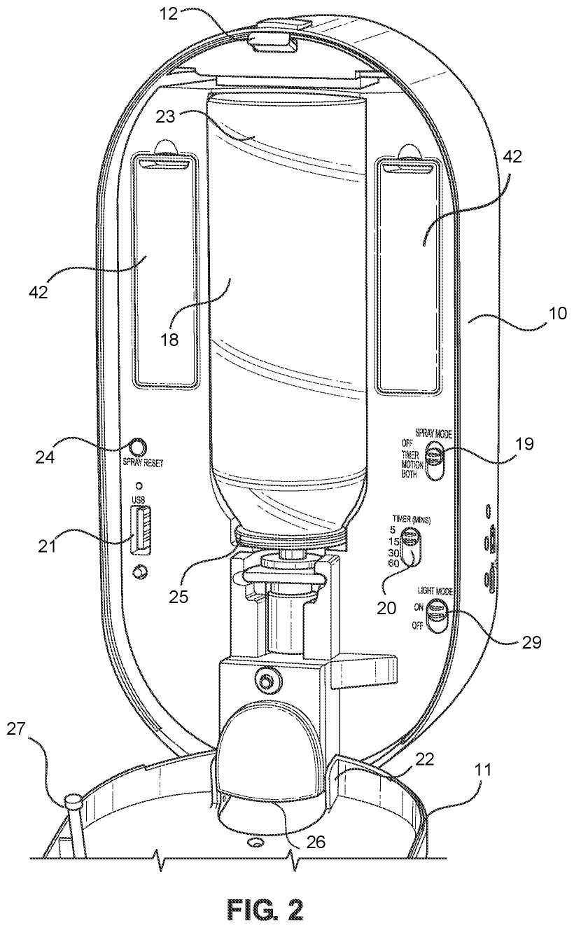

Referring now to FIG. 1, there is shown a perspective view of the exterior of an embodiment of the door handle sanitizer. The door handle sanitizer generally includes a housing 10 having a rear surface that is mountable to a door. The mounting to the door can be accomplished via an adhesive material or via any other suitable fastening mechanism. An access lid 11 is pivotally attached to the housing and configured to move between a closed position covering an interior volume of the housing (shown in FIG. 1) and open position exposing the interior volume of the housing (shown in FIG. 2). In the shown embodiment, a latch 12 is configured to selectively secure the access lid in the closed configuration. The latch 12 may include a spring or other biasing mechanism, such that depressing the latch 12 releases the lid 11 from the closed position. In some embodiments, the latch 12 includes a locking mechanism that is operably connected to the lid 11 or the latch 12. This may include a key lock or other suitable locking mechanism with a unique access key. In such embodiments, the latch 12 is configured to open to release the access lid 11 only when the locking mechanism receives the unique access key.

The dispensing mechanism includes an outlet nozzle portion 16 that dispenses sanitizing material onto the door handle. In the shown embodiment, a dispenser indicator light 17 is configured to illuminate when the dispensing mechanism actuator is activated and the door handle is sprayed with sanitizer, which can help boost the confidence or peace of mind of the user by indicating that the sanitizing action is occurring. The device may include additional indicator lights for informational purposes. For example, the shown embodiment includes a battery replacement indicator light 13 operably connected to the power supply, which is configured to illuminate when a battery power supply needs to be replaced or recharged. As another example, the shown embodiment also includes a spray refill light 14 which is operably connected to a spray reset button (shown in FIG. 2). The spray refill light 14 is configured to illuminate to indicate to the user that the internal container of sanitizing material must be replaced or refilled. Further, the shown embodiment includes a photosensor 15 disposed on the housing 10 that detects ambient light in the room. The photosensor 15 can be used in different ways to control operation of the door handle sanitizer. For example, the photosensor 15 can be utilized to trigger a low power or non-operational state if the room in which the device is in use is currently unoccupied, which can save energy and save sanitizing material.

Referring now to FIG. 2, there is shown a perspective view of the interior of an embodiment of the door handle sanitizer. A sanitizer refill container 18 is removably secured with an interior volume of the housing 11, which is accessible when the lid 12 is opened. The sanitizer refill container 18 holds sanitizing material to be dispensed on to the door handle. In one embodiment, the sanitizing material is composed of a combination of hypochlorous acid, sodium chloride, and water. However, other sanitizing solutions may be utilized. The sanitizer refill container 18 can be replaced with a new container 18 when it is emptied, or may be refilled with material and reinserted. In the shown embodiment, the sanitizer refill container 18 is removably secured within a slot 23 that is recessed into a front surface of the interior of the housing 10. This allows the sanitizer refill container 18 to be easily removed and replaced as needed.

The dispensing mechanism is configured to be removably connected to an open lower end 25 of the sanitizer refill container 18. This can be done via a threaded connection, a snap-fit connection, a gasket or friction fit, or any other suitable connection that allows easy insertion and removable of the sanitizer refill container 18. The connecting portion 28 allows the interior volume of the refill container 18 to be in fluid communication with the outlet nozzle 26 of the dispenser mechanism. In this way, the nozzle 26 is configured to dispense a material from the sanitizer refill container 18 when the dispensing mechanism is activated.

The interior volume of the housing 10 also includes various control mechanisms for controlling the operation of the door handle sanitizer. In the shown embodiment, the spray mode control 19 can select between timer-operated spray, motion-activated spray, both types of activations, or no activations. The timer interval selection 20 allows the user to select the duration of the time interval between activations of the dispenser. The shown embodiment also includes an indicator switch 29 that controls the operation of the dispensing indicator light. Additionally, the shown embodiment includes a spray reset button 24 that can be manually activated to reset an internal mechanism for monitoring flow of the material or the amount of material remaining in the refill container 18. In some embodiments, a contact member 27 disposed on the access lid 11 is configured to depress a spray reset button 24 within the interior volume when the access lid 11 is closed.

A power supply, such as a battery, powers the device. The battery can be recharged via an onboard electronic port such as a USB port 21. The battery can also be a replaceable battery stored within a battery compartment 42. The power supply is operably connected to a motion sensor, a timer, a plurality of indicator lights, the actuator of the dispensing mechanism, and a control circuit. In some embodiments, the USB port 21 can be operably connected to the control circuit in order to receive usage data regarding the number of times the dispenser has been activated or other information.

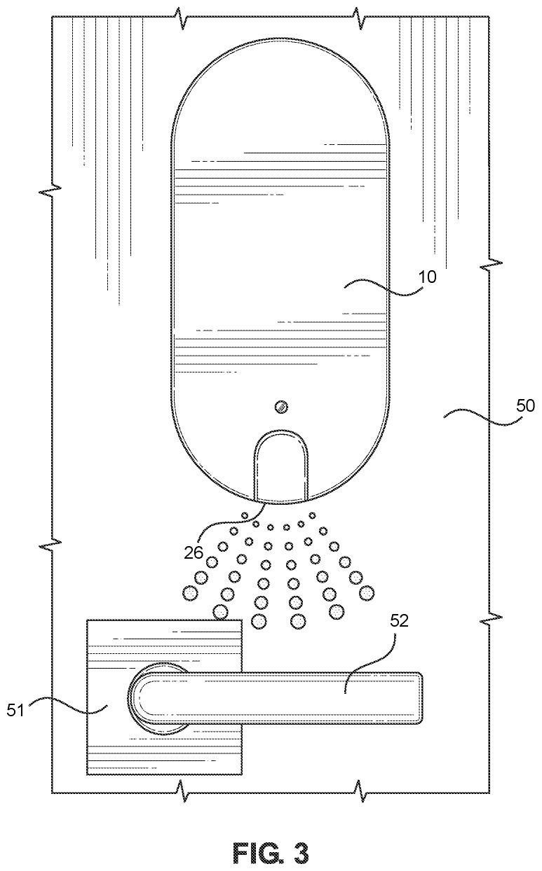

Referring now to FIG. 3, there is shown a front elevation view of an embodiment of the door handle sanitizer attached to a door and sanitizing the door handle. The housing 10 is secured to the door 50 just above the handle 51. The nozzle 26 is configured to dispense sanitizing material in a spray pattern that spreads to cover the grasping portion 52 of the door handle 51. The device is shown in use with a handle, but it is capable of being used with many of door knob types or other projections extending outwardly from the door surface, such as a push bar or other mechanism that individuals typically grasp when using the door, such as a fixed handle.

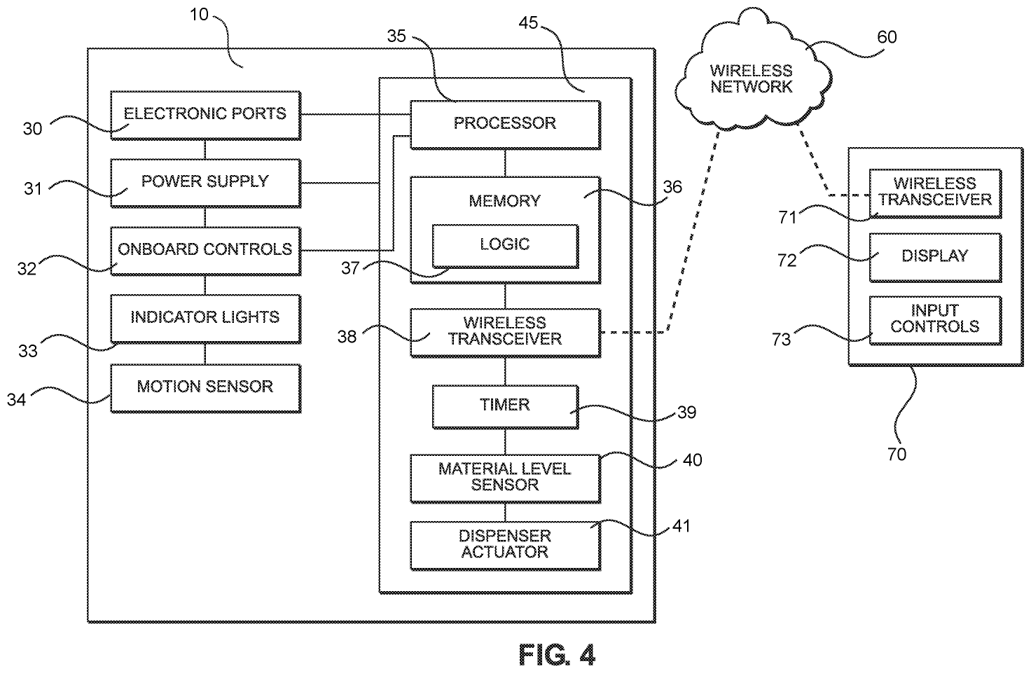

Referring now to FIG. 4, there is shown a block diagram of the various components of an embodiment of the door handle sanitizer. The housing 10 includes the electronic ports 30, the power supply 31, the onboard controls 32, the indicator lights 33, and a motion sensor 34, all of which are operably connected. The motion sensor 34 may be a photosensor or any other sensor that can detect motion proximal to the dispensing outlet. The onboard controls 32, including the timer interval control, the spray mode control, and the light control are all operably connected to a control circuit 45. The control circuit 45 includes a processor, a non-transitory computer readable medium operatively connected to the processor, and a logic stored in the non-transitory computer readable medium that, when executed by the processor, causes the system to perform a method. If the spray mode switch is set to a timer interval selection, then the dispensing mechanism actuator 41 is activated consecutively over a time interval. The time internal is controlled via a timer 39 such as an internal timing circuit or the like. In some embodiments, a material level sensor 40 such as an optical sensor or any other suitable sensor can be utilized to measure the current level of material in the refill container. In other embodiments, it can be utilized to track the output of the material through the nozzle of the dispenser.

In operation, the user can control when the dispenser is activated via the onboard controls 32. As previously mentioned, the spray mode switch can be set to a timer interval selection, such that the dispensing mechanism actuator 41 is activated consecutively over a time interval. If the spray mode switch is set to a motion activation selection, then the dispensing mechanism actuator 41 is activated when the motion sensor 34 detects motion. If the spray mode switch is set to a combination time interval and motion activation selection, then the dispensing mechanism actuator 41 is activated consecutively over a time interval and also when the motion sensor detects motion. In this way, the user can customize the operation of the sanitizer to suit their personal preferences.

In some embodiments, the control circuit 45 further includes a wireless transceiver 38, which is configured to send and receive information via a wireless network 60. This allows the control circuit 45 to receive command instructions from a remote device 70 via a wireless network 60. The wireless network 60 may include an internet connection, a wireless LAN, a bluetooth connection, or any other suitable wireless communication protocols. In such embodiments, the operation of the door handle sanitizer may be monitored by and controlled by the remote device 70 that is also in wireless communication with the wireless network 60 via its own wireless transceiver 71. The remote device 70 may include a smartphone, computer, tablet, or any other suitable device that includes a display 72 for displaying information and an input control 73 for inputting and transmitting command instructions. For example, some embodiments allow the user to remotely select the operational mode, overriding the onboard controls 32. Further, the wireless transceiver 38 can be utilized to transmit usage information as determined by the material level sensor 40 or other means. This information can be received by the remote device 70, allowing the user to monitor the use of the door handle sanitizer via the remote device's display 72.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, and all equivalent relationships to those illustrated in the drawings and described in the specification are intended to be encompassed by the present invention. Therefore, the foregoing is considered as illustrative only of the principles of the invention. Further, it is not desired to limit the invention to the exact construction and operation shown and described, and accordingly, all suitable modifications and equivalents may be resorted to, falling within the scope of the invention.

I claim:

1) A door handle sanitizer comprising:
a housing having a rear surface that is mountable to a door;
an access lid pivotally attached to the housing and configured to move between a closed position covering an interior volume of the housing and open position exposing the interior volume of the housing;
a dispensing mechanism comprising a connector disposed within the interior volume that is configured to removably secure an open end of a sanitizer refill container thereto and a nozzle configured to dispense a material from the sanitizer refill container when the dispensing mechanism is activated;
a power supply operably connected to a motion sensor, a timer, a plurality of indicator lights, an actuator of the dispensing mechanism, and a control circuit;
the control circuit comprising a spray mode switch, a processor, a non-transitory computer readable medium operatively connected to the processor, and a logic stored in the non-transitory computer readable medium that, when executed by the processor, causes the system to perform a method, the method comprising:
if the spray mode switch is set to a timer interval selection, then activating the dispensing mechanism actuator consecutively over a time interval;
if the spray mode switch is set to a motion activation selection, then activating the dispensing mechanism actuator when the motion sensor detects motion; and
if the spray mode switch is set to a combination time interval and motion activation selection, then activating the dispensing mechanism actuator consecutively over a time interval and activating the dispensing mechanism actuator when the motion sensor detects motion.

2) The door handle sanitizer of claim 1, further comprising a latch configured to selectively secure the access lid in the closed configuration.

3) The door handle sanitizer of claim 2, further comprising a locking mechanism operably connected to the latch, wherein the latch is configured to open to release the access lid only when the locking mechanism receives a unique access key.

4) The door handle sanitizer of claim 1, wherein the motion sensor comprises a photosensor.

5) The door handle sanitizer of claim 1, further comprising an electronic port operably connected to the power supply.

6) The door handle sanitizer of claim 5, wherein the electronic port comprises a USB port disposed within the interior volume.

7) The door handle sanitizer of claim 1, wherein the timer interval is determined via a time interval selection switch that is operably connected to the timer.

8) The door handle sanitizer of claim 1, wherein the power supply comprises one or more batteries disposed in one or more battery compartments within interior volume.

9) The door handle sanitizer of claim 8, wherein the plurality of indicator lights comprises a battery replacement indicator light operably connected to the power supply.

10) The door handle sanitizer of claim 1, further comprising a spray reset button operably connected to a spray refill light of the plurality of indicator lights.

11) The door handle sanitizer of claim 1, wherein the plurality of indicator lights further comprises a dispenser indicator light that is configured to illuminate when the dispensing mechanism actuator is activated.

12) The door handle sanitizer of claim 1, wherein the spray mode switch is disposed within the interior volume of the housing.

13) The door handle sanitizer of claim 1, further comprising a wireless transceiver configured to receive command instructions from a remote device via a wireless network.

14) The door handle sanitizer of claim 1, further comprising a slot disposed within the interior volume that removably receives the sanitizer refill container.

15) The door handle sanitizer of claim 1, further comprising a contact member disposed on the access lid that is configured to depress a spray reset button within the interior volume when the access lid is closed.
